(19) United States
(12) Patent Application Publication
(10) Pub. No.: US 2017/0215741 A1
(43) Pub. Date: Aug. 3, 2017

(54) DEVICE FOR MEASURING THE TEMPERATURE IN THE ANUS OR VAGINA OF AN ANIMAL

(71) Applicant: SMARTBOW GMBH, Weibern (AT)

(72) Inventor: Wolfgang AUER, Weibern (AT)

(73) Assignee: SMARTBOW GMBH, Weibern (AT)

(21) Appl. No.: 15/328,698

(22) PCT Filed: Jul. 28, 2015
(86) PCT No.: PCT/AT2015/000104

§ 371 Date: Jan. 24, 2017

(30) Foreign Application Priority Data
Jul. 31, 2014 (AT) A 610/2014
Sep. 22, 2014 (AT) A 717/2014

(51) Int. Cl. A61B 5/01; G01J 5/02; G01K 13/00; A61B 5/00

(52) U.S. Cl. CPC A61B 5/01; A61B 5/4255; A61B 5/4337; A61B 5/0008; A61B 5/0086; G01J 5/0205; G01K 13/004; A61B 2503/40; A61B 2090/034

(57) ABSTRACT

The invention relates to a device for measuring the temperature in the anus or in the vagina of an animal, wherein a front longitudinal part, an abutment part and a rear longitudinal part lie in succession on the device, wherein the front longitudinal part is equipped with a temperature sensor and, when used as intended, it is intended to be inserted into the anus or the vagina of the animal until the abutment part approximately rests against the outside of the body of the animal. One or more further temperature sensors are attached to the rear longitudinal part and/or to the abutment part, which, when used as intended, are provided for measuring the temperature in the surroundings of the animal.

WO 2013188895 A2 describes a method and a system for recording data in a data network, said data relating to manipulations, for example temperature measurements, performed on an animal. The animal, the manipulation appliance, and any person performing the manipulation, are registered in a data network and are each equipped with electronic devices which are able to transmit a unique identification signature, wherein the transmitted identification signatures are received by a central data collection site. Parts of the transmission path of the identification signatures, transmitted as electrical signals, are the body of the person performing the manipulation and the body of the animal on which the manipulation is performed.

The documents DE 694 25 492 T2, WO 2010/054312 A1, U.S. Pat. No. 5,792,070 A, U.S. Pat. No. 6,347,039 81 and WO 2009045868 A1 disclose devices of the kind in question, that is to say devices for measuring the temperature in the anus or in the vagina of an animal. The devices basically have a slender, elongate design. When used as intended, the front longitudinal area of these devices, which is equipped with a temperature sensor, is inserted to a certain depth into the anus or the vagina. The rear longitudinal area remains protruding outside the body opening. At the boundary between the front longitudinal part and rear longitudinal part, the devices have a thickened area, or a part jutting out in the transverse direction. By means of the device being inserted into the body opening until this thickened area or this jutting-out part rests on the outer surface of the animal, the depth of insertion, is defined exactly, which is advantageous for the reproducibility of the measurement and also for avoiding pain or injury. The measured temperature can either be read off on the part of the device remaining outside the body of the animal, or lines lead away from this part to an evaluation and/or data collection unit.

In the case of a manipulation, the transmission of the identification signature can be started automatically by the manipulator touching the animal in question or by the used manipulation appliance (e.g. clinical thermometer) touching the animal in question. The central data collection site can thus automatically record which person has performed a manipulation on which animal, and with which appliance and at which time. Apart from the fact that the clinical thermometer used has to be ready for said data transmission, the clinical thermometer used is not defined in any more detail.

The object of the invention is to improve a device for measuring the temperature in the anus or in the vagina of an animal by comparison with the devices of this kind that have been discussed, and to do this in such a way as to improve the meaningfulness of the measurement results obtained concerning the condition of the animal on which the measurement has been performed.

To achieve said object, the invention proceeds from the known construction in which the device has an elongate, slender design and, when the device is used as intended, a longitudinal part equipped with a temperature sensor is intended to toe inserted into the anus or the vagina of the animal, and wherein another longitudinal part extends out of the body opening, and wherein the device, at the boundary between the two longitudinal parts, has an abutment part which rests on the outside of the animal and thus prevents the device from sliding too far into the body opening of the animal.

As an improvement according to the invention, it is proposed that the device has a second temperature sensor, namely on that longitudinal part which, when the device is used as intended, does not extend into the body opening of the animal, this second temperature sensor being provided for measuring the surrounding temperature.

When measuring the body temperature with the first (inner) temperature sensor, it is possible to use the second (outer) temperature sensor to measure and record the surrounding temperature at a defined distance from the animal. Since the surrounding temperature is also known when measuring the body temperature, the measurement result with regard to the body temperature is more meaningful than would be the case if the surrounding temperature were not known. The body temperature in fact fluctuates to a slight extent and also, with a (variable) time delay, along with fluctuations of the surrounding temperature. For the purpose of a more accurate assessment of the state of health or state of fertility of the animal, this concurrent fluctuation of the body temperature with fluctuations of the surrounding temperature can be largely disregarded.

In a preferred embodiment, the device is equipped with electronics which are able to transmit the measured results by radio, such that a remote receiving station is able to receive the results and store them without a human being having to act as an “intermediate data carrier”.

In another preferred embodiment, the device itself has a unique identification signature which is stored using data technology and which it is able to transmit as well as the measurement results, such that it is also possible, at the receiving side, to automatically record, with respect to the measurement results, with exactly which appliance the temperature measurements have been carried out.

In another preferred embodiment, the device has an electrode on the surface that comes into contact with the animal when the device is used as intended, by way of which electrode electrical signals are exchangeable, with the body of the animal serving as conducting medium for electrical signals. It is thus possible to use the body of the animal as part of the transmission path of electrical signals that have been output from the device. Moreover, it is thus easily possible to automatically detect contact of the device with the animal, since all that has to be done for this purpose is to check, with the aid of a further radio node, whether a signal transmission via the body of the animal is possible or not.

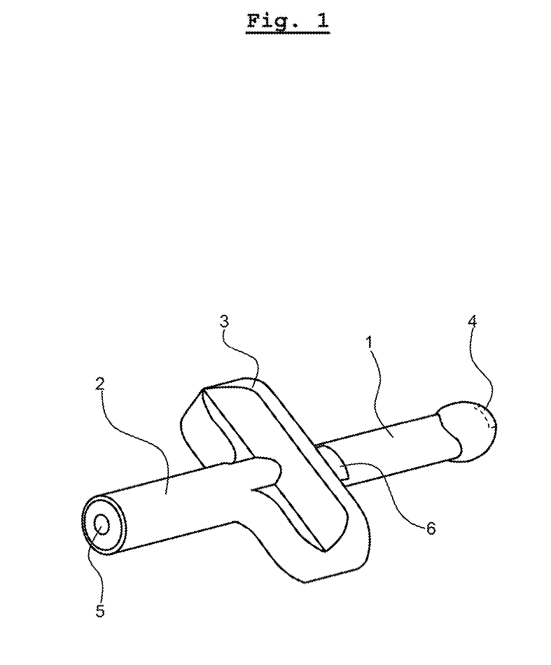

FIG. 1: shows a perspective view of an example of a device according to the invention.

The device according to the invention, in the example shown in FIG. 1, basically has:
a front (inner) longitudinal part 1 which, when used as intended, is inserted into the anus or vagina of an animal in order to measure the temperature,
a rear (outer) longitudinal part 2 which, when used as intended, extends away from the animal,
an abutment part 3 which forms a widening in relation to the front longitudinal part 1, such that, when the longitudinal part 1 is inserted into the body opening of the animal f the abutment part 3 comes to rest on the outside of the animal and forms a stop to prevent the device from being inserted too far.

In the front part of the longitudinal part 1, a first temperature sensor 4 is attached which, when used as intended, serves to measure the internal body temperature of the animal. The temperature sensor can be, for example, a temperature-dependent electrical resistor or a thermocouple. In a preferred embodiment, it is an infrared radiation sensor arranged behind a radioparent disk. This is preferable for the reason that sensors of this kind measure particularly quickly, and they are therefore able to give a usable measurement result even in fractions of a second.

The second temperature sensor which is intended to measure the surrounding temperature outside the animal, is attached to the end of the rear longitudinal part directed away from the animal when the device is used as intended.

In the advantageous embodiment shown, the front longitudinal part 1, near its end area directed toward the rear longitudinal part 2, is provided with an electrode surface 6 which, upon contact with the body of the animal, is able to transmit electrical signals between the body of the animal and electronics (not shown) contained in the device. The electrode surface 6 can therefore be used not only for the data transmission, via the body of the animal as described above, but also as a sensor surface with which it is possible to determine whether the front longitudinal part 1 is inserted (substantially) completely into the body opening of the animal.

Of course, a sensor that detects complete insertion can also be realized by other means, for example with the aid of a pressure switch which is arranged on the abutment part 3 or just in front of the latter on the longitudinal part 1.

A further electrode surface (not shown), required for the radio transmission of data to and from the device, is preferably an electrically conductive surface effective as an antenna, which is arranged as far as possible on the end area of the rear longitudinal part 2 directed away from the front longitudinal part 1.

The electronics (not shown) contained in the device operate the temperature sensors, optionally read out the identification signature of the device, and control the transmission and reception of radio signals to/at a data collection station.

In an alternative configuration to the configuration shown in FIG. 1, a temperature sensor 5, preferably based on infrared radiation measurement, can be attached to each of the two free end areas of the abutment part, the detector surface of which temperature sensor 5 does not come directly into contact with the animal when the device is used as intended. By using two temperature sensors 5 and by logical evaluation of the results, measurement errors can be better avoided than would be the case when using only one temperature sensor.

At the free end of the rear longitudinal part 2, if is then possible, for example, to provide an opening for a compartment for an exchangeable battery.

On account of the speed that can be achieved, the one or more temperature sensors 5, which measure the surrounding temperature when used as intended, are likewise preferably based on the known principle of infrared radiation measurement. For this purpose, the surface of the sensor directed toward the surroundings is a layer, typically of plastic, which is as thin as possible, while still having sufficient mechanical strength, and which, on account of its thinness, picks up the surrounding temperature very quickly. The radiation-sensitive measuring surface of the sensor arranged in the interior of the longitudinal part 2 or 3 is oriented such that it specifically receives the radiation that radiates inward from the thin layer.

In a preferred development, the device contains a signal transmitter which, by means of the electronics contained in the device, is controlled to transmit a signal when the temperature measurement is finished. As a signal, a light can light up, a sound can be emitted, or a vibration device can briefly vibrate.

In a preferred development, the front longitudinal part 1 is equipped with a sensor device which measures the depth of insertion of the longitudinal part 1 into the body opening of the animal, even when the longitudinal part is not fully inserted. This is important when, for whatever reason, it is not practicable or not possible to insert the front longitudinal part 1 for temperature measurement completely into the body opening of the animal. In an advantageous embodiment, which is based on measurement by capacitively conducted electrical currant, this sensor is embodied as a series of electrode surfaces arranged along the longitudinal part, the electrodes being arranged just underneath the surface of the longitudinal part but being separated from the surroundings by an electrical insulating layer. The electrodes are controlled with high-frequency alternating voltage. The measurement principle is based on the fact that capacitive current flow between the electrode and the surroundings is greater or lesser depending on whether the surface of the front longitudinal part 1 on which an electrode is located is open to air or is within a body opening of the animal.

Of course, other measurement principles are also conceivable for measuring the depth of insertion. For example, a series of optical sensors, which measure the brightness, can be arranged behind transparent disks along the jacket surface of the front longitudinal part 1. Instead of optical sensors, pressure sensors can also be arranged which detect the mechanical pressure exerted on the front longitudinal part 1 by the inside of the animal.

Moreover, it is also possible, for example, to determine the depth of insertion of the front longitudinal part 1 on the basis of the ohmic resistance between electrical contacts, for which purpose electrical contacts are placed along the surface of the longitudinal part 1, and, when the longitudinal part 1 has been inserted, electrical contact takes place between the electrical contacts via the body areas of the animal that lie on them.

One or more sensor surfaces which detect whether the longitudinal part 1 is inserted completely into the body opening of the animal can also be arranged on the side of the abutment part 3 directed toward the longitudinal part 1. These sensor surfaces can also function according to the above-described measurement principle, which is based on capacitively conducted electrical current.

However, they can also be configured, for example, in the form of a pressure sensor, light sensor, temperature sensor or ohmic sensor.

In a preferred embodiment, the device is equipped with an acceleration sensor, and the electronics contained in the device are designed to detect a characteristic acceleration profile and, as a result of the latter, to start a temperature measurement procedure. Since it is thus possible to do without switches or buttons for starting a temperature measurement procedure, the device can be more easily designed to be completely sealed and therefore less susceptible to soiling.

1. A device for measuring the temperature In the anus or in the vagina of an animal, wherein a front longitudinal part, an abutment part and a rear longitudinal part lie in succession on the device, wherein the front longitudinal part is equipped with a temperature sensor and, when used as intended, it is intended to be inserted into the anus or the vagina of the animal until the abutment part approximately rests against the outside of the body of the animal,
wherein
one or more further temperature sensors are attached to the rear longitudinal part and/or to the abutment part and, when used as intended, are provided for measuring the temperature in the surroundings of the animal.

2. The device as claimed in claim 1, wherein it contains an electronic circuit which is able to transmit the results of the temperature measurements by radio.

3. The device as claimed in claim 1, wherein it contains an electronic circuit in which an identification signature of the device is stored in such a way that it can be read out and/or can be transmitted.

4. The device as claimed in claim 2, wherein a surface area which comes into contact with the animal, when the front longitudinal part is inserted into the animal, has an electrode surface through which electrical signals are exchangeable, with the body of the animal serving as conducting medium for electrical signals.

5. The device as claimed in claim 4, wherein the electrode surface is arranged on the end area of the end area of the front longitudinal part directed toward the rear longitudinal part.

6. The device as claimed in claim 1, wherein the temperature sensor is an infrared radiation sensor.

7. The device as claimed in claim 1, wherein it has a sensor with which the completion of insertion of the longitudinal part into the body opening of the animal can be detected.

8. The device as claimed in claim 1, wherein it has a sensor which, even when the longitudinal part is not fully inserted, is able to measure how deep the longitudinal part is inserted into the anus or the vagina of the animal.

9. The device as claimed in claim 1, wherein it has a signal transmitter, by means of which the end of the measuring procedure can be signaled.

10. The device as claimed in claim 1, wherein the one or more sensors are based on the measurement principle of measurement of infrared radiation.

11. The device as claimed in claim 1, wherein it is equipped with an acceleration sensor, and in that a temperature measurement procedure can be started in accordance with measurement results of the acceleration sensor.
